(19) United States
(12) Patent Application Publication
(10) Pub. No.: US 2025/0009615 A1
(43) Pub. Date: Jan. 9, 2025

(54) Method for Improving an Athletes Gripping Performance and Deterring Perspiration

(71) Applicant: Yousif A Saroya, Astoria, NY (US)

(72) Inventor: Yousif A Saroya, Astoria, NY (US)

(21) Appl. No.: 18/814,972

(22) Filed: Aug. 26, 2024

Related U.S. Application Data
(63) Continuation-in-part of application No. 17/288,304, filed on Apr. 23, 2021, now Pat. No. 11,233,539.
(63) Continuation-in-part of application No. 16/372,665, filed on Apr. 2, 2019, now abandoned.
(60) Provisional application No. 62/652,799, filed on Apr. 4, 2018.

(51) Int. Cl. A61K 8/26; A61K 8/02; A61K 8/19; A61Q 15/00

(52) U.S. Cl. CPC A61K 8/26; A61K 8/0225; A61K 8/19; A61Q 15/00; A61K 2800/412

(57) ABSTRACT

A method for reducing human perspiration and/or enhancing an athlete's grip. The method comprises: (a) providing an oxide-free powder composition containing at least about 65% of a chalk component and about 10-35% of an aluminum chlorohydrate-based additive then (b) applying said powder composition to designated areas of the human body, i.e., the underarms for perspiration reduction and the hands and feet for gripping enhancement.

### CROSS REFERENCE TO RELATED APPLICATION

This application is a continuation-in-part of pending U.S. application Ser. No. 17/228,304, filed on Apr. 12, 2021, which was a continuation-in-part of pending U.S. application Ser. No. 16/372,665, filed on Apr. 2, 2019, which itself was a perfection of Provisional U.S. Application No. 62/652,799, filed on Apr. 4, 2018, all disclosures of which are fully incorporated herein.

### BACKGROUND OF THE INVENTION

### 1. Field of the Invention

This invention relates to an improved composition for athletic use. More particularly, it relates to an improved chalk-based composition that is oxide-free, wax-free and sawdust-free while exhibiting better perspiration deterrence AND improved gripping for certain sports particularly rock and wall climbing. It is meant for applying to human skin on one or more of: the hands, feet, arms, underarms, groin and other body areas. It may be applied as either a powder, solid stick, roll on or spray.

### 2. Relevant Art

Disclosures pertinent to the present invention include, but are not limited to, the following references: U.S. Pat. Nos. 4,536,413, 4,572,690, 6,656,257, 7,201,914, US Published Application No. 2004/019728, US Published Application No. 2006/0045860, US Published Application No. 2012/0321579, US Published Application No. 2016/0338917, PCT Application No. WO03005981, European Patent Application No. 1080715 and European Patent Application No. 1175885.

The present invention is directed to a composition (and method of use) for controlling, preventing, ameliorating and/or treating perspiration contamination on the skin of human hands, arms, underarms, feet, groin and other body areas. It will also reduce or control the symptoms and side effects of such perspiration, like reduced gripping for certain athletic activities.

Some undesirable skin maladies are associated with conditions that promote or allow unchecked growth of bacteria on or within perspiring skin. For example, acne occurs when increased production of skin lipids causes the pores of the skin to close or be blocked. This causes further skin secretions (primarily perspiration and lipids) to back up within the pores. Normal flora can then proliferate in such pores, fueled by the body's own secretions of moisture (perspiration) and fuel (lipids). The body responds to this mounting bacterial load and build-up of skin secretions with an inflammatory reaction, producing the hallmark acne lesions.

Other skin conditions are also produced or exacerbated by the combination of microbial infection, chronic moisture and the host's response, including tinea pedis (athlete's foot), tinea cruris (jock itch), psoriasis, seborrhea and eczema.

It has been observed that aluminum salts, especially those related to aluminum chlorohydrate, can inhibit the production of the perspiration and lipids. When applied to the human skin, these compounds form a colloidal plug or gel that prevents release of perspiration and lipids. Since these fluids are not released, the body does not produce more. This not only keeps the skin dry, but also prevents microbes on the skin from feeding off of the fluids and damaging the skin.

Accordingly, it is an object of the present invention to overcome prior perspiration prevention failures and to provide a new composition (and related method of use thereof) that will control, prevent, ameliorate, or otherwise treat undesirable topical (i.e. outer skin) perspiration on the hands, arms, underarms, feet, groin, and other body areas of its users.

It is desired to use a certain size of aluminum chlorohydrate particle with the present invention, particularly one that is about 100 microns or less.

In an earlier filing, claims were rejected over a combination of references that included both Savanuck and Sereboff. It is now respectfully argued herein that a reliance on both references is inappropriate. Savanuck taught powder compositions comprising an inorganic oxide in admixture with an astringent of aluminum chlorohydrate present in an amount ranging from about 1% to 25%. Savanuck applied that composition to hands for an athlete's improved grip, his astringent improving the friction enhancing properties of his oxides by reducing perspiration which was absorbed by and deactivated the friction enhancing properties of his oxides.

But Savanuk never expressly taught combining a magnesium carbonate component with aluminum chlorohydrate. Savanuck only focused on oxides. Magnesium carbonate (MgCO₃) is unequivocally not an oxide. The definition of an oxide in chemistry is a compound composed of oxygen atoms bonded to another element. For instance, magnesium oxide (MgO) fits this definition perfectly, with magnesium bonded solely to oxygen. On the contrary, magnesium carbonate contains not just magnesium and oxygen but also carbon. Its chemical formula, MgCO₃, clearly indicates the presence of carbon alongside magnesium and oxygen. This difference in composition is fundamental and indisputable, making magnesium carbonate distinctly a carbonate compound and not an oxide.

To make up for that difference, it was argued that Sereboff taught compositions comprising particulate magnesium or calcium carbonate (i.e., chalk) in a frictional grip enhancing composition that would provide improved frictional grip properties and moisture absorption. Further, Sereboff allegedly suggested using silicates or oxides as an alternative to magnesium and/or calcium carbonate to achieve similar improvement in frictional properties. However, magnesium carbonate and silica are fundamentally distinct compounds due to their elemental composition. Magnesium carbonate comprises magnesium, carbon, and oxygen atoms, whereas silica consists of silicon and oxygen atoms. This elemental variance results in differing chemical properties and functionalities that are crucial for understanding their respective applications in skincare formulations.

Silica is commonly utilized in skincare products for its ability to improve texture, absorb excess oil, and provide a smooth feel on the skin. Conversely, magnesium carbonate is known for its absorbent and drying properties, making it suitable for applications such as chalk for enhanced grip in activities like rock climbing or gymnastics. While both compounds may contribute to skincare formulations, they serve distinct functions and cannot be interchangeably used to achieve friction-enhancing effects on the skin. Silica and magnesium are more like opposites rather than substitutes for one another.

### SUMMARY OF THE INVENTION

The present invention is directed to a method of using a new composition that serves as an improved antiperspirant agent for better controlling, preventing, ameliorating, or otherwise treating the undesirable perspiration of a human's hands, arms, feet, groin and other body areas. It will also reduce or control symptoms and side effects of such perspiration like reduced gripping for certain athletic (and/or work) activities. One embodiment of the present invention comprises combining a known topical dessicant, i.e., chalk, with one main additive (preferably aluminum chlorohydrate) such that the effects of such combination are superior to the use of either active component alone.

### DETAILED DESCRIPTION OF PREFERRED EMBODIMENTS

The present invention comprises a composition and method for controlling, preventing, ameliorating, or otherwise treating undesirable topical perspiration on the skin of human hands and feet. It may also be used on other body areas including the arms, underarms, feet and/or groin of its users to reduce or control the symptoms/side effects of such perspiration. A key feature of the present invention is a combination of a known topical antiperspirant and a special additive (in preferred quantities), wherein the effects of such combination are superior to the use of either active component alone. The term antiperspirant is defined herein as an agent that checks, reduces or eliminates perspiration.

Rock climbing is an outdoor and indoor sport in which athletes ascend natural rock cliffs, faces, boulders as well as artificial gym climbing holds with the goal of not falling off the rock face. In order to ascend, climbers rely on their grip/frictional contact to tactfully move from hold to hold. Perspiration slickens the skin on the hands thus reducing friction and grip ability.

Climbers routinely use chalk (magnesium carbonate, calcium carbonate, and other chalk compounds) to absorb perspiration to enhance grip/frictional contact on rocks and climbing holds. At times of increased perspiration (due to any amount of circumstances e.g. increased environmental temperature, humidity, mental stress, etc.) a climber must constantly reapply chalk to maintain optimal grip/friction. The act of applying chalk during the midst of climbing is taxing on the body (one must let go of the rock face with one hand to reapply, while the other hand does twice the work to continue to hold onto the rock formation). This ultimately leads to either a more taxing/strenuous ascent or complete failure to ascend due to wasted energy spent on chalk reapplication instead of the act of climbing. This invention introduces an antiperspirant agent (a type of aluminum salt) to limit/suppress perspiration. This in turn limits the need for chalk reapplication, thus improving product efficacy and ultimately increasing climbing ability.

### Preferred Formulation

A base component of magnesium carbonate with an addition of about 5-35% of aluminum chlorohydrate, preferably about 10-30% and most preferably about 15-25%. An ideal mixture would be an 80-20 combination of the two main components.

Other chalk compounds such as calcium carbonate may be substituted or added as well as other aluminum-based antiperspirant compounds, especially aluminum sesquichlorohydrate and/or aluminum zirconium tetrachlorohydrate.

Still other substitute ingredients for aluminum chlorohydrate include: aluminum chloride, aluminum chloride hexahydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium glycine, aluminum zirconium pentachlorohydrex gly, aluminum zirconium tetrachlorohydrex gly, aluminum zirconium trichlorohydrate and/or aluminum zirconium trichlorohydrex glycine.

Why certain ingredients and ranges were excluded:

A range of 5-35% was chosen due to these reasons: (1) test subjects could not notice the effects of the antiperspirant in formulations that contained less than 5% antiperspirant. (2) at or above 5% subjects experienced reduced perspiration. And (3), at above 35%, the texture of the formulation started to exhibit a grittier texture that reduced friction and grip ability.

Rosin was tested as an additional grip enhancer, but was ultimately not included in the formulation due to the fact that this ingredient left a sticky/tacky residue on climbing holds and the skin itself.

Silica powder was considered as an additional perspiration absorber, but further research showed that if inhaled, silica powder can cause Silicosis (scarring of lung tissue), increased risk of TB, lung cancer, and other forms of lung disease.

Talc and corn starch were also tested as additional perspiration absorbers. These ingredients, however, reduced friction and gripping ability due to their slick/slippery texture.

The powder composition is also wax-free and sawdust-free, unlike some of the prior art known compositions. In fact, this composition is free of an assortment of other materials/compounds as well. Though they may not be expressly “listed” here, the invention is also FREE of most other known or subsequently developed additives in OTHER prior art compositions-even those not currently known to Applicant or his counsel at this time.

### EXAMPLES

### Efficacy Test:

Objective: to determine/quantify the sweat reduction properties of Aluminum Chlorohydrate/Magnesium Carbonate powder blends in ratios of 1) 10/90, 2) 15/85, and 3)

Hypothesis: As Aluminum Chlorohydrate has been proven to exhibit antiperspirant properties (sweat reduction or suppression), the higher Aluminium Chlorohydrate to Magnesium Carbonate blends will showcase less sweat production.

### Materials and Method:

Aluminum Chlorohydrate powder, preferably about 100 microns or less in size
Magnesium Carbonate powder
Webril Handi-Pads (square cotton pads)
Brifit Professional Digital Mini Scale
Medical tape
Scissors
Spatula
Cosmetic containers (3×)
Plastic wrap
   1) Cut and measure 5 equally sized and weighted cotton pads
   2) Cut and measure 10 equally sized and weighted medical tape strips
   3) Blend one batch of 10/90, 15/85, and 20/80 weighted powder mixes of Aluminum Chlorohydrate/Magnesium Carbonate and place into 3 separate, labelled cosmetic containers.
      3a) 10/90=“A”, 15/85=“B”, and 20/80=“C”
   4) Measure equal portions of A, B, C, and 100% Magnesium Carbonate powder and place on separate cotton pads
      4a) Empty/clean cotton pad=“D” (Control), and 100% Magnesium Carbonate powder=“E”
   5) Wipe test subject's lower back clean with a clean cotton pad
   6) Place cotton pads A, B, C, D, and E, product side down, touching skin, and affix tape to the top and bottom of each cotton pad
   7) Wrap lower back area with plastic wrap to minimize sweat evaporation and secure cotton pads and product to skin.
   8) Have subject sweat with exercise, increase room temperature and/or humidity, for at least 40 mins
   9) Take each pad off and weigh (pad, remaining product, and 2 pieces of tape)
      9a) 1 cotton pad, product, and 2 pieces of tape=“package”
   10) To measure the amount of sweat produced, subtract the weight of the After (+sweat) package from the Before (no sweat) package, and note the total difference

### Results

A and B weighed the same, totaling 0.03 g of sweat
C weighted the least of the group, totaling 0.01 g of sweat
D and E weighted equally and the most, totaling 0.04 g of sweat

### Qualitative Observations:

A: skin felt moist and slippery
B and C: skin felt dry to the touch
D: skin felt the wettest of the group
E: skin felt wet and pasty

### CONCLUSION

Compared to the Control (D), the 10% and 15% Aluminum Chlorohydrate powder blends (A & B) reduced sweat by 25%; one can extrapolate that the results hold for the entire 10-15% range.

The 20% Aluminum Chlorohydrate powder blend (C) reduced sweat by 75%.

100% Magnesium Carbonate (E, market standard product) does not have any antiperspirant properties, thus produced equal amounts of sweat as the Control (D, no product).

As climbing is a tactile sport, qualitative results are also important:

Though the 15% Aluminum Chlorohydrate powder blend only reduced sweat by 25% versus the 75% reduction observed with the 20% Aluminum Chlorohydrate powder blend, the 15% blend nevertheless exhibited a desired dry grip enhancing feel on the skin. This dry grip enhancing feel was even more prominent in the 20% Aluminum Chlorohydrate powder blend.

Both the 15% powder blend (B) and 20% powder blend (C) can be seen as desirable ratios, the latter being the more aggressive blend. Compared to the market standard of 100% Magnesium Carbonate powder (E), the 10-20% Aluminum Chlorohydrate blends introduced sweat reduction, and improved tactile feel and grip.

The examples and various powder applications are provided for illustrative purposes, as the present invention is not limited to the recited examples and applications and includes other modalities and formulations known to those skilled in the art.

Any combined antiperspirant/gripper composition of the present invention may comprise, for example, one or more additives for making a powder alternative that is more of a viable liquid suspension. For use in gloves, shoes, athletic equipment, and similar items, the composition may be applied to the relevant skin areas prior to donning such items, or may be applied to the “apparel” items themselves (i.e., the gloves, etc.) prior to their donning. Such composition may be added to such items at the time of manufacture (for example, inside protective gloves during manufacture). In some instances (such as on the hands), it is preferable that the composition comprises a liquid that does not contain moisturizing components, so as to avoid loss of dexterity or grip.

Moreover, one or more of the active ingredients may be encapsulated into a time-release vehicle (e.g., liposome or similar vehicle) to enhance its longevity of efficacy.

Having described the presently preferred embodiments, it is to be understood that this invention may otherwise be covered by the scope of the claims that follow.

What is claimed is:

1. A method for improving an athlete's gripping performance, said method comprising:
(a) providing an oxide-free, rosin-free, sawdust-free powder composition comprising:
(i) at least about 65% of a magnesium carbonate chalk component combined with
(ii) about 10-35% of an aluminum chlorohydrate; and
(b) applying the powder composition to the athlete's hands or feet.

2. The method of claim 1 wherein the powder composition contains about 85% magnesium carbonate and about 15% aluminum chlorohydrate.

3. The method of claim 1 wherein the powder composition contains about 80% magnesium carbonate and about 20% aluminum chlorohydrate.

4. The method of claim 1 wherein the powder composition contains about 75% magnesium carbonate and about 25% aluminum chlorohydrate.

5. The method of claim 1 wherein the aluminum chlorohydrate is up to about 100 microns in particle size.

6. A method for deterring human perspiration, said method comprising:
(a) providing an oxide-free, rosin-free, sawdust-free powder composition comprising:
(i) at least about 65% magnesium carbonate combined with
(ii) about 10-35% aluminum chlorohydrate; and
(b) applying the powder composition to an external area of the human body prone to perspiring.

7. The method of claim 6 wherein the external area of the human body includes the underarms.

8. The method of claim 6 wherein the powder composition contains about 85% magnesium carbonate and about 15% aluminum chlorohydrate.

9. The method of claim 6 wherein the powder composition contains about 80% magnesium carbonate and about 20% aluminum chlorohydrate.

10. The method of claim 6 wherein the powder composition contains about 75% magnesium carbonate and about 25% aluminum chlorohydrate.

11. The method of claim 6 wherein the aluminum chlorohydrate is up to about 100 microns in particle size.

12. A method for deterring perspiration on an external area of a human body, said method comprising:
(a) providing a composition that is oxide-free, wax-free, sawdust-free, rosin-free, silica powder-free, talc-free, and corn starch-free, said composition comprising:
(i) at least about 65% magnesium carbonate combined with
(ii) about 10-35% of an aluminum chlorohydrate-based component selected from the group consisting of: aluminum chlorohydrate, aluminum sesquichlorohydrate, aluminum zirconium tetrachlorohydrate and combinations thereof; and
(b) applying the composition to the external areas of the human body prone to perspiring.

13. The method of claim 12 wherein the composition is provided in a powder form for application to the external area of the human body.

14. The method of claim 12 wherein the aluminum chlorohydrate component consists essentially of an aluminum chlorohydrate powder having a particle size of up to 100 microns.

15. The method of claim 12 wherein the composition contains about 85% magnesium carbonate and about 15% of the aluminum chlorohydrate-based component.

16. The method of claim 12 wherein the composition contains about 80% magnesium carbonate and about 20% of the aluminum chlorohydrate-based component.

17. The method of claim 12 wherein the composition contains about 75% magnesium carbonate and about 25% of the aluminum chlorohydrate-based component.
